(19) United States
(12) Patent Application Publication
(10) Pub. No.: US 2009/0220590 A1
(43) Pub. Date: Sep. 3, 2009

(54) Triple drug combinations for psychiatric patients

(76) Inventor: Ikemefuna Nkanginieme, Canton, OH (US)

Correspondence Address: Ikemefuna Nkanginieme M.D., 4910 Staffordshire Court, Canton, OH, 44718, US

(21) Appl. No.: 12/384,451

(22) Filed: Apr. 6, 2009

(51) Int. Cl. A61K 9/48

(52) U.S. Cl. 424/452

(57) ABSTRACT

The mega capsule can be prescribed by any physician, and in particular those professionals in the psychiatric field. The mega capsule will be a form of medication designed for a specific patient in mind. The capsule will help the patients understand their medications better and make it easier for them to take. The patient may only have to take the mega capsule once or twice a day and by doing that, the patient will feel more in control of their prescriptions. By inventing the mega capsule, we may lower the cost significantly for the patients. Altogether, it will make life easier for the patients and the physicians.

### CROSS-REFERENCE TO RELATED APPLICATIONS

Not applicable

### FEDERALLY SPONSORED RESEARCH

Not applicable

### SEQUENCE LISTING OF PROGRAM

Not applicable

### BACKGROUND OF INVENTION

My invention consists of a triple drug combination (mega capsule) designed for use by medical professionals on patients who are suffering from various psychiatric problems that require a combination of medications. The patients in mind for the mega capsule consist of millions of patients with co-morbid disorders including anxiety disorders, mood disorders, and psychotic disorders.

From my experience with the patients receiving these medications individually, they complain of being on too many pills and often have difficulties with compliance. They would rather have one prescription written out for them rather than three individual prescriptions. Patients on multiple medications often feel over medicated and would rather take only one single prescription preferably as a once single dose or at the most, twice daily doses. Some patients also have difficulties remembering which medications to take and when specifically to take them. Multiple medications and multiple directions could cause medication errors and sometimes lead to inadvertent overdoses.

### BRIEF SUMMARY OF INVENTION

This invention will combine three medications already in use into one mega capsule to improve compliance which could also improve efficacy profile. The pills are chosen, not only for their relatively favorable side effects, but are also combined in such a way as to minimize the various side effects of each individual medication for the patients involved. If patients only need to take one pill once or twice during their day, it will make them more independent and they hopefully will be happier about the medications they are taking. The directions involved for taking this mega capsule would be much more clear and-concise to the patients than taking multiple prescriptions. Additional target of the three drug combinations is a possible reduction in side effects such as weight gain, sexual disturbances, and metabolic disturbances.

The mega capsule used for the patients mentioned would include the following combinations of medications: (Geodon+Wellbutrin+Ativan); (Geodon+Wellbutrin+Klonopin); (Geodon+Wellbutrin+Xanax); (Geodon +Wellbutrin+Valium); (Geodon+Wellbutrin+Cogentin); (Geodon+Lexapro+Ativan); (Geodon +Lexapro+Klonopin); (Geodon+Lexapro+Xanax); (Geodon+Lexapro +Valium); (Geodon+Lexapro+Cogentin); (Geodon+Deplin+Ativan); (Geodon+Deplin+Klonopin); (Geodon+Deplin+Xanax); (Geodon+Deplin+Valium); (Geodon+Deplin+Cogentin); (Geodon+Cymbalta+Ativan); (Geodon+Cymbalta+Xanax); (Geodon+Cymbalta+Klonopin); (Geodon+Cymbalta+Valium); (Geodon+Cymbalta+Cogentin); (Abilify+Wellbutrin+Ativan); (Abilify+Wellbutrin+Klonopin); (Abilify+Wellbutrin+Xanax); (Abilify+Wellbutrin+Valium); (Abilify+Wellbutrin+Cogentin); (Abilify+Lexapro+Ativan); (Abilify+Lexapro+Klonopin); (Abilify+Lexapro+Xanax); (Abilify +Lexapro+Valium); (Abilify+Lexapro+Cogentin); (Abilify+Deplin+Ativan); (Ativan+Deplin+Klonopin); (Abilify+Deplin+Xanax); (Abilify +Deplin+Valium); (Abilify+Deplin+Cogentin); (Abilify+Cymbalta+Ativan); (Abilify+Cymbalta+Xanax); (Abilify+Cymbalta+Klonopin); (Abilify+Cymbalta+Valium); (Abilify+Cymbalta+Cogentin).

The possibility of lowering cost for the patient could be a real advantage. A large majority of patients have co-pays that are required for every filled prescription and that co-pay is necessary before receiving the medications. For instance, if a patient has a 25 dollar co-pay for every prescription, they need to come up with 75 dollars. By combining the three medications into one mega capsule or one prescription, instead of three, the patient would only need to have the 25 dollars necessary for their single co-pay. For obvious reasons, the patient would prefer the 25 dollar scenario to the 75 dollar. The invention of the mega capsule would instantly start to save patients money.

The scales that would be used to measure the success of the mega capsules may include the HAMD, Beck's anxiety and depression scales, the AIMS, and various compliance/satisfaction scales. The length of study would be about three months and the study would contain approximately 50-150 subjects. The subjects permitted to be enrolled into the study would need to have one of the three following disorders or combination of these disorders: 1. Psychotic disorders, 2. Mood disorders, and 3. Anxiety disorders.

### BRIEF DESCRIPTION OF THE DRAWINGS

The drawings included are informal drawings of the mega capsule exterior and interior. The chemical structures have also been included into the drawings to show the components needing to be combined in order to make the capsules.

### DETAILED DESCRIPTION OF THE INVENTION

The capsule shown in FIG. 1 is the image of the first option of the design of the mega capsule. FIG. 1 shows the three chambers of the capsule where the medication would be kept separate. FIG. 2 is the view of the second option, the mixture of the three medications. For example, Abilify 1a, Wellbutrin 2b, and Ativan 3c would make-up the inside of the capsule as a mixture with no chambers.

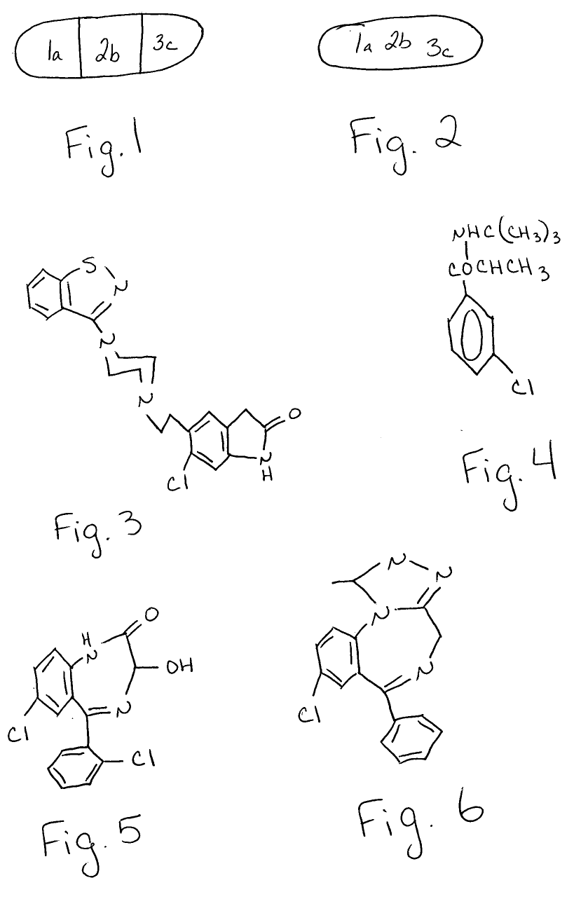

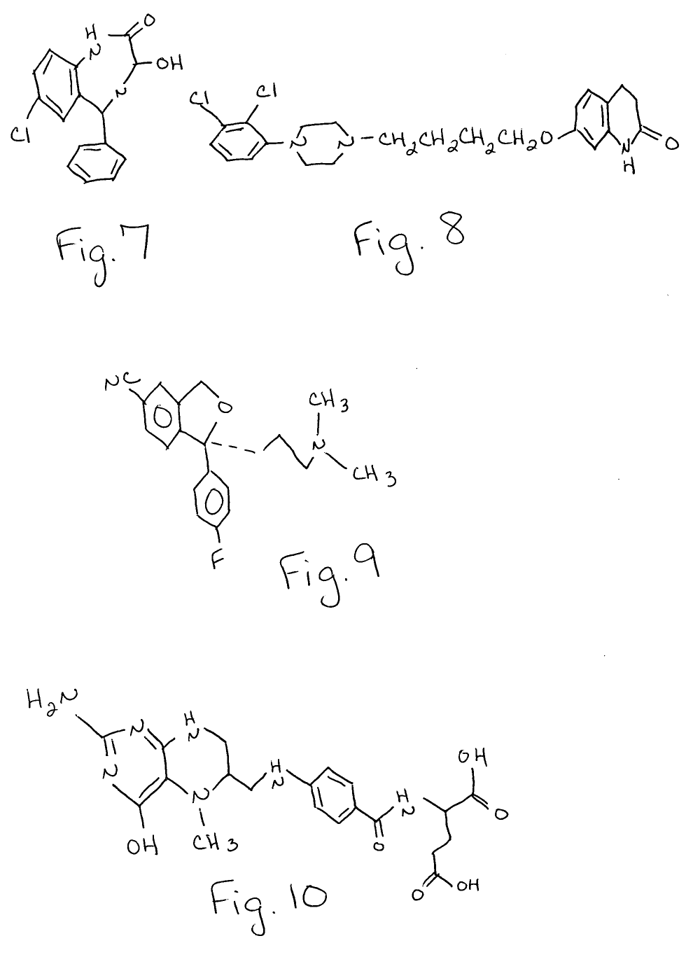

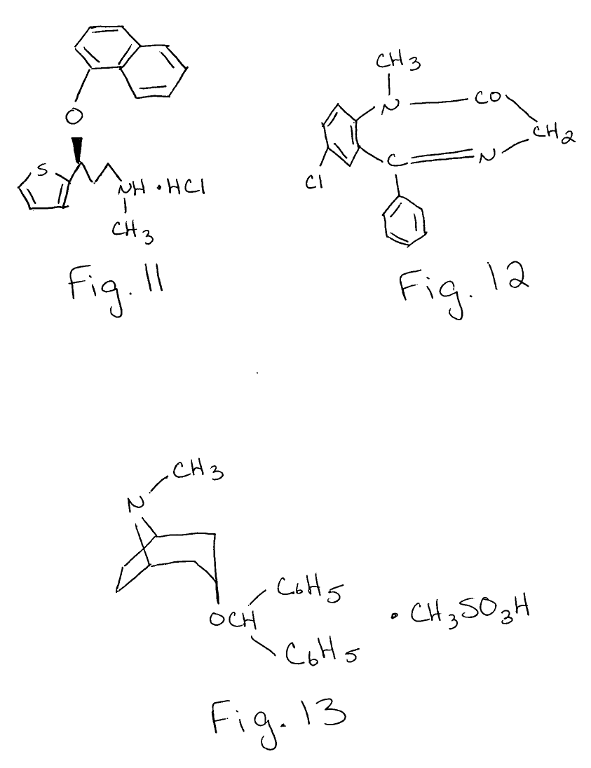

The chemical structures of the individual medications are also listed in the drawings; please refer to FIG. 3 thru FIG. 13. In FIG. 3, we have the chemical structure of Geodon that would be combined with FIG. 4 Wellbutrin and FIG. 5 Ativan to create the mega capsule.

I claim:

1. The mega capsule will be made into a cylindrical shape that contains either three chambers or one chamber containing all three drugs.

2. The medication combinations will be added together inside the mega capsule of claim 1.

3. A number of three medications will be used to fill the capsule of claim 1.

4. There will be forty different combinations of medications.

5. The said combinations will consist of the medications Abilify, Geodon, Lexapro, Wellbutrin, Deplin, Cymbalta, Xanax, Ativan, Klonopin, Valium, and Cogentin.

6. The said medication combinations will vary based upon the milligrams of each.
